(19) United States
(12) Patent Application Publication
(10) Pub. No.: US 2021/0352932 A1
(43) Pub. Date: Nov. 18, 2021

(54) METHODS OF PRODUCING PLANT PROTEIN FROM FOOD WASTE USING MICROALGAE

(71) Applicant: Sophie's BioNutrients Pte. Ltd., Singapore (SG)

(72) Inventor: Yao-Hsin Wang, Singapore (SG)

(73) Assignee: Sophie's BioNutrients Pte. Ltd., Singapore (SG)

(21) Appl. No.: 15/930,720

(22) Filed: May 13, 2020

(51) Int. Cl. A23J 1/00; B01D 11/02; B01D 21/26; A23J 3/34; C12P 21/00

(52) U.S. Cl. CPC A23J 1/009; B01D 11/0288; C12P 21/00; A23J 3/347; B01D 21/262

(57) ABSTRACT

Methods to extract chlorella protein from algae powder are described. A first extraction method is an alkaline solution extraction method. A second extraction method is an enzyme extraction method. A third method is a low-temperature deep eutectic solvents (DES) extraction method. According to each of these methods, a protein recovery rate is calculated from a protein extract solution of chlorella protein.

### FIELD OF THE EMBODIMENTS

The field of the invention and its embodiments relate to methods to extract chlorella protein from algae powder. In particular, the present invention introduces a first alkaline solution extraction method, a second enzyme extraction method, and a third low-temperature deep eutectic solvents (DES) extraction method.

### BACKGROUND OF THE EMBODIMENTS

Chlorella is a genus of single-celled green algae belonging to the division Chlorophyta. Chlorella I spherical in shape, about 2 to 10 μm in diameter, and is without flagella. It contains the green photosynthetic pigments chlorophyll-a and -b in its chloroplast. Chlorella multiples rapidly, requiring only carbon dioxide, water, sunlight, and a small amount of minerals to reproduce.

Chlorella is a potential food source since it is high in protein and other essential nutrients. For example, when dried, chlorella contains about 45% protein, 20% fat, 20% carbohydrate, 5% fiber, and 10% minerals and vitamins. Due to this, chlorella has been labeled as a “superfood” and has garnished significant attention from the vegan community. Further, chlorella has been explored as a potential source of food and energy because its photosynthetic efficiency can, in theory, reach 8%, which exceeds that of other highly efficient crops, such as sugar cane.

With increasing attention being paid to the consumption of healthy nutritional foods, algal protein has moved to the forefront of non-animal protein sources. However, the applications of chlorella protein as a functional ingredient in food still requires further exploration. Compared to the protein of other crops, chlorella protein and its extraction is a relatively unstudied subject. Algae protein is usually extracted by mechanical grinding, high-pressure homogenization, ultrasonic treatment, pulse dyslenoid to release the protein molecules to facilitate further extraction processes like water, alkali or enzyme, and then use of isoelectric precipitation, and salting out (salt induced precipitation) methods. To date, however, protein extraction methods for chlorella have limited commercial use due to the scale up failures.

Thus, a need exists for an improved method of producing plant protein from food waste using microalgae that provides a low-cost, high chlorella protein extraction rate suitable for industrial application.

### Review of Related Technology:

U.S. Pat. No. 8,835,142 B2 describes a method to process biomass (e.g., plant biomass, animal biomass, microbial, and municipal waste biomass) to produce useful products, such as food products and amino acids.

WO 2015/071908 A1 describes a method to produce microalgae that shows high growth rate under wide conditions, including extreme light intensities.

WO 2007/134294 A2 describes algal species and compositions, as well as methods for identifying algae that produce high lipid content, possess tolerance to high CO₂, and/or can grow in wastewater.

U.S. Published Patent Application No. 2003/0211594 A1 describes a novel microalgal strain and progeny thereof, useful for the remediation of waste water.

U.S. Published Patent Application No. 2018/0155227 A1 describes a biorefinery system (BIOSYS) that effectively treats all human activity-derived waste (e.g., black water, grey water, and food waste streams) using biological systems and produces as process by-products: recovered potable water, liberated free oxygen, edible protein cake (with and without lipids), soil amendments, and machinery lube oils.

WO 2009/086307 A1 describes a method for treating biomass waste to result in usable byproducts. Biomass is treated to remove debris, transferred to microbial digester units, such as anaerobic and aerobic digesters, and the resultant solids and liquids are provided to an algae production unit. Algae are harvested and beneficial byproducts are retained. Gases, heat and energy produced by energy conversion units are used in units of the system or provided to external sources. Water is cleaned and when separated from the algae and other solids in the algae harvesting unit may be provided to external sources, or may be used in other units of the system.

CN 105861312 A describes a method for culturing microalgae by adding an anaerobic digestion liquid of kitchen waste into natural seawater, aims to find out the best proportion, and belongs to the technical field of the microalgae. According to the invention, the digestion liquid is added to natural seawater according to the ratio of (1:10)-(1:50) to be taken as an experimental group culture medium, BG11, natural seawater and the digestion liquid are taken as a control group, the experimental group culture medium is cultured under the condition of continuous light until the microalgae stops to grow, and centrifugal separation is carried out to obtain the microalgae. The result shows that the growth rate of the microalgae added with the digestion liquid and cultured in natural seawater is obviously higher than that of the microalgae cultured in the BG 11 and pure seawater, furthermore, natural seawater added with the digestion liquid is taken as the culture medium to improve the lipid yield of the microalgae and lower the cultivation cost of the microalgae, so that the method for culturing the microalgae by adding the anaerobic digestion liquid of the kitchen waste to natural seawater is worthy of being popularized and applied.

Various methods to extract chlorella protein are known in the art. However, their means of operation are substantially different from the present disclosure, as the other inventions fail to solve all the problems taught by the present disclosure. The present invention and its embodiments provide a first alkaline solution extraction method, a second enzyme extraction method, and a third low-temperature deep eutectic solvents (DES) extraction method.

### SUMMARY OF THE EMBODIMENTS

The present invention and its embodiments provide methods to extract chlorella protein from algae powder. In particular, the present invention introduces a first alkaline solution extraction method, a second enzyme extraction method, and a third low-temperature deep eutectic solvents (DES) extraction method.

A first embodiment of the instant invention describes an extraction method for chlorella protein. The method includes adding an alkaline solution to algae powder to form a mixture. The algae powder is present in a range of approximately 4.98 grams to approximately 5.02 grams. The algae powder comprises a protein content in a range of approximately 60% to approximately 65%. In some examples, the protein content of the algae powder is approximately 61.51%. The alkaline solution is approximately 1% to approximately 8% of a weight of the chlorella protein. In some examples, the alkaline solution is a sodium hydroxide (NaOH) solution.

The method also includes extracting the chlorella protein from the mixture at a temperature of approximately 50° C. for a time period of approximately 6 hours. The method then includes centrifuging the mixture at approximately 800 rpm for a time period of approximately 20 minutes to obtain a protein extract solution of the chlorella protein. Next, the method includes calculating a protein recovery rate from the protein extract solution of the chlorella protein. The protein recovery rate may be calculated based on the following equation:

$$Protein recovery rate / \% \frac{Supernatant protein content \times supernatant mass}{Chlorella mass \times algal powder protein content} \times 100$$

A second embodiment of the instant invention describes an enzyme extraction method for chlorella protein. The method includes dissolving approximately 25.0 grams of an algae powder in approximately 375 mL of water to form a solution. The method also includes adding an alkaline protease in a range of approximately 0.01% to approximately 0.2% to the solution.

The method includes adjusting the pH of the solution to a pH of 8.0. Next, the method includes hydrolyzing the solution at a temperature of approximately 55° C. with an alkaline solution for a time period of approximately 24 hours. The method further includes centrifuging the mixture for a time period of approximately 20 minutes to obtain a protein extract solution of the chlorella protein. The method also includes calculating a protein recovery rate from the protein extract solution of the chlorella protein.

A third embodiment of the instant invention describes a low-temperature deep eutectic solvent (DES) extraction method of chlorella protein. The method includes: adding a first material:a second material having molar ratios of 1:2 to algae powder:cryogenic co-melt solvent having molar ratios of 1:9 to form a mixture. In examples, the first material:second material is glycerol:choline chloride. In other examples, the first material:second material is urea:choline chloride.

The method also includes reacting the mixture at a temperature of approximately 60° C. for a time period of approximately 3 hours. Next, the method includes centrifuging the mixture for a time period of approximately 20 minutes to obtain a protein extract solution of the chlorella protein. The method further includes: calculating a protein recovery rate from the protein extract solution of the chlorella protein.

In general, the present invention succeeds in conferring the following benefits and objectives.

It is an object of the present invention to provide a low-cost method for extracting chlorella protein.

It is an object of the present invention to provide a method yielding a high-protein extraction rate for chlorella protein.

It is an object of the present invention to provide a method for extracting chlorella protein having use in the industrial sector.

It is an object of the present invention to provide an alkaline solution extraction method for chlorella protein.

It is an object of the present invention to provide an enzyme extraction method for chlorella protein.

It is an object of the present invention to provide a low-temperature deep eutectic solvent (DES) extraction method for chlorella protein.

### BRIEF DESCRIPTION OF THE DRAWINGS

FIG. 1 depicts a schematic block diagram of a traditional alkaline solution extraction method for chlorella protein, according to at least some embodiments described herein.

FIG. 2 depicts tabular results of the traditional alkaline solution extraction method for chlorella protein of FIG. 1, according to at least some embodiments described herein.

FIG. 3 depicts tabular results of pre-treating chlorella by defatting with ethanol, according to at least some embodiments described herein.

FIG. 4 depicts tabular results of reducing a material:liquid ratio and extending a reaction time from FIG. 3, according to at least some embodiments described herein.

FIG. 5 depicts tabular results of obtaining a supernatant following centrifugation, according to at least some embodiments described herein.

FIG. 6 depicts tabular results of obtaining a supernatant following centrifugation, according to at least some embodiments described herein.

FIG. 7 depicts tabular results of obtaining a supernatant following centrifugation, according to at least some embodiments described herein.

FIG. 8 depicts a graph showing an amount of sodium hydroxide (NaOH) added as compared to a protein yield, according to at least some embodiments described herein.

FIG. 9 depicts tabular results of adding an increased amount of solid sodium hydroxide (NaOH) to chlorella to increase a protein yield percentage, according to at least some embodiments described herein.

FIG. 10 depicts tabular results of adding an increased amount of solid sodium hydroxide (NaOH) to chlorella to increase a protein yield percentage, according to at least some embodiments described herein.

FIG. 11 depicts tabular results of increasing the reaction time period from FIG. 10, according to at least some embodiments described herein.

FIG. 12 depicts tabular results of a protein yield percentage from varying an amount of sodium hydroxide in a range between 1% to 8%, according to at least some embodiments described herein.

FIG. 13 depicts a schematic block diagram of an enzyme extraction method for chlorella protein, according to at least some embodiments described herein.

FIG. 14 depicts tabular results of the enzyme extraction method of FIG. 13, according to at least some embodiments described herein.

FIG. 15 depicts tabular results of the enzyme extraction method of FIG. 13, according to at least some embodiments described herein.

FIG. 16 depicts a schematic block diagram of a first low-temperature deep eutectic solvents (DES) extraction method for chlorella protein, according to at least some embodiments described herein.

FIG. 17 depicts a schematic block diagram of a second low-temperature deep eutectic solvents (DES) extraction method for chlorella protein, according to at least some embodiments described herein.

### DESCRIPTION OF THE PREFERRED EMBODIMENTS

The preferred embodiments of the present invention will now be described with reference to the drawings. Identical elements in the various figures are identified with the same reference numerals.

Reference will now be made in detail to each embodiment of the present invention. Such embodiments are provided by way of explanation of the present invention, which is not intended to be limited thereto. In fact, those of ordinary skill in the art may appreciate upon reading the present specification and viewing the present drawings that various modifications and variations can be made thereto.

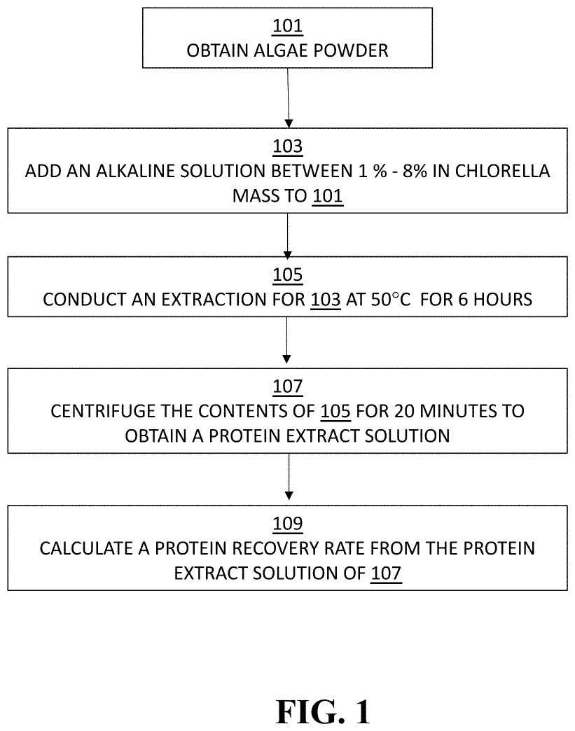

FIG. 1 depicts a schematic block diagram of a traditional alkaline solution extraction method for chlorella protein, according to at least some embodiments described herein. Since most proteins are acidic, when a protein is near its isoelectric point (PI—4 to 5), solubility will be minimized. For example, in alkaline conditions, proteins will be more soluble. At the same time, alkaline affects the protein molecule's secondary bonds. Hydrogen bonds can have a certain destructive effect and can change the polarity, so that the protein molecular surface charge changes, which modifies the solubility of the protein molecules, which will separate the protein for extraction.

The method of FIG. 1 begins at a process step 101, which involves obtaining algae powder. In some examples, the algae powder is a green bao algae powder comprising a protein content in a range of approximately 60% to approximately 65%. In some examples, the protein content is approximately 61.51%. The mass of chlorella may be in a range of approximately 4.98 grams to approximately 5.02 grams.

The process step 101 is followed by a process step 103, which involves adding an alkaline solution to the algae power to form a mixture. In examples, the amount of the alkaline solution is in a range between approximately 1% to approximately 8% of a weight of the chlorella protein, or in the range between approximately 0.050 grams to approximately 0.40 grams. In other examples, the alkaline solution is a sodium hydroxide (NaOH) solution.

The process step 103 is followed by a process step 105, where an extraction of the mixture of the process step 103 is carried out at approximately 50° C. for approximately 6 hours. The process step 105 is followed by a process step 107, where the contents of the process step 105 are centrifuged at approximately 800 rpm for approximately 20 minutes to obtain a protein extract solution of the chlorella protein. The process step 107 is followed by a process step 109, where a protein recovery rate is calculated from the protein extract solution of the chlorella protein from the process step 107. The protein recovery rate may be calculated by Equation 1 shown below:

$$Protein recovery rate / \% \frac{Supernatant protein content \times supernatant mass}{Chlorella mass \times algal powder protein content} \times 100 \qquad [ Equation 1 ]$$

It should be appreciated that the method of FIG. 1 may also be used to extract soy protein, pea protein, sea buckthorn protein, and other plant proteins from different materials not explicitly listed herein.

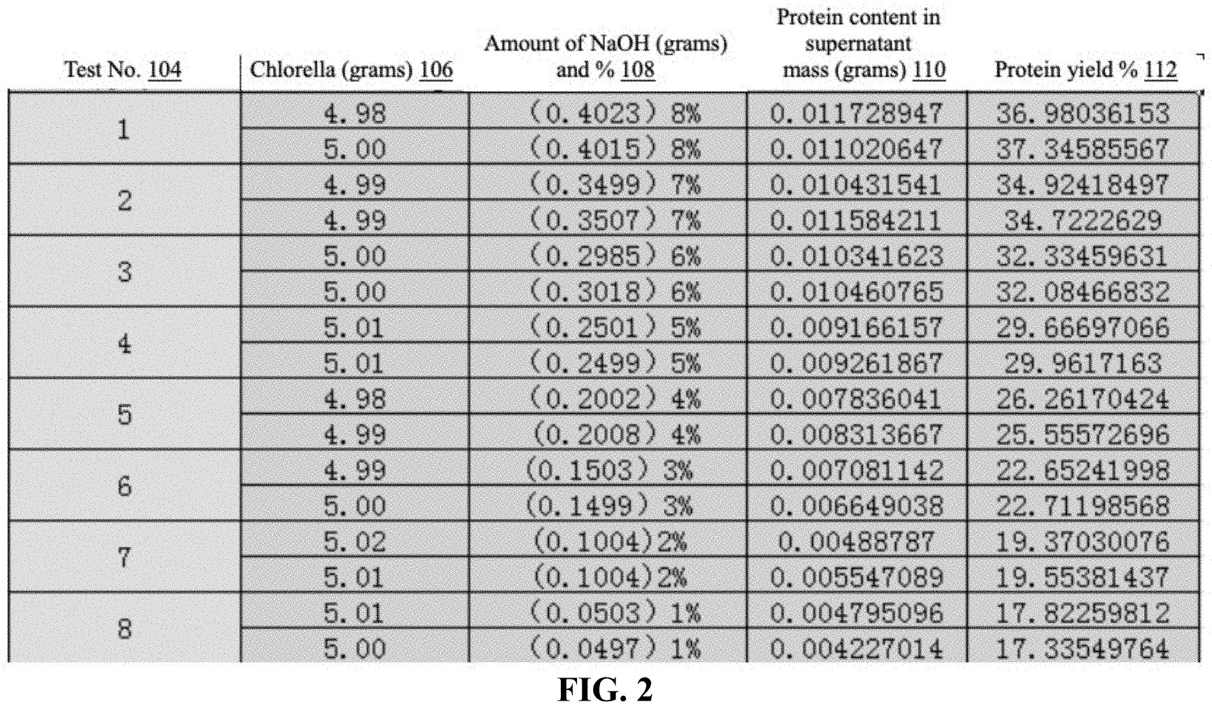

FIG. 2 depicts tabular results of the traditional alkaline solution extraction method for chlorella protein of FIG. 1, according to at least some embodiments described herein. The tabular results of FIG. 2 from the method of FIG. 1 include numerous columns and rows. The columns include the following: a test number column 104, a chlorella column 106 (which is an amount of the chlorella measured in grams), an amount of the NaOH solution column 108 (measured in both grams and a %), a mass of a protein content in supernatant column 110 (measured in grams), and a protein yield % column 112 (calculated by Equation 1). The rows represent numerous tests conducted by varying these parameters.

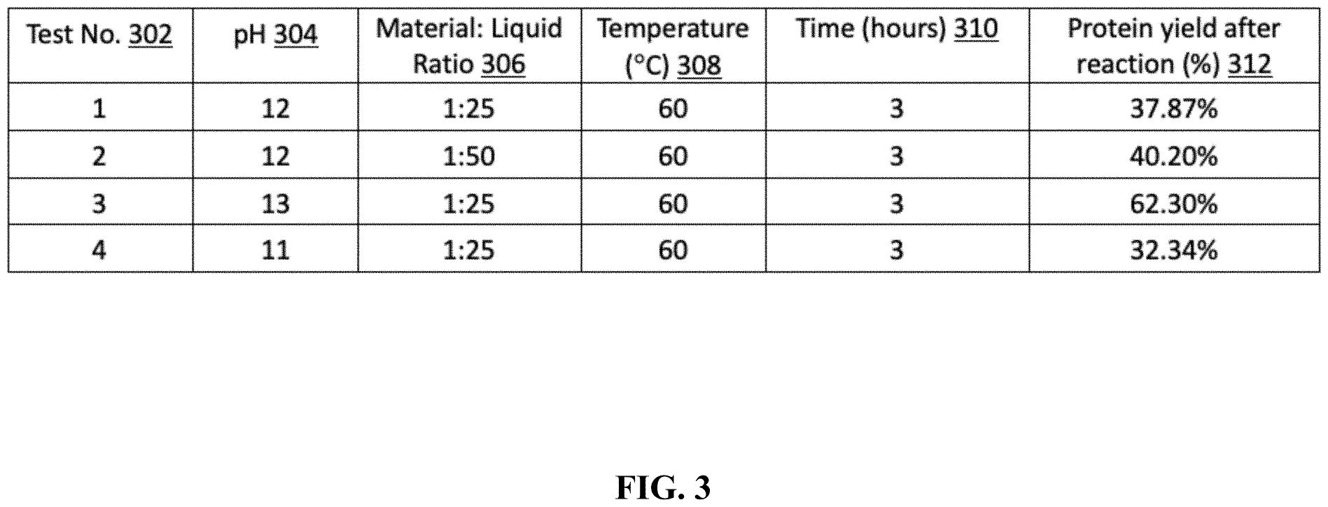

FIG. 3 depicts tabular results of pre-treating chlorella by defatting with ethanol, according to at least some embodiments described herein. Similar to FIG. 2, the tabular results of FIG. 3 include numerous rows and columns. The columns include, at least, a test number column 302, a pH column 304, a material:liquid ratio column 306, a temperature column 308 (which is measured in ° C.), a reaction time period column 310 (which is measured in hours), and a protein yield column 312 (which is measured as a percentage after the reaction). In examples, the pH of the pH column 304 can range from a pH of 11 to a pH of 13. In further examples, the material:liquid ratio can range from 1:25 to 1:50.

The chlorella may be pretreated without breaking the wall, which may occur prior to the process step 101 of FIG. 1. The chlorella may be defatted with ethanol on a shaker at a specific material:liquid ratio, at a specific temperature, and for a specific time period. A single factor test may then be performed at various pH values and various material:liquid ratios. The protein content in the supernatant may then be measured.

As an illustrative example (described as Test 1), the chlorella may be defatted with the ethanol on the shaker at the material:liquid ratio of 1:25, at the temperature of approximately 60° C., and for the time period of approximately 3 hours. A single factor test may then be performed at a pH of 12. The protein content in the supernatant may then be measured as 37.87%.

As another illustrative example (described as Test 3), the chlorella may be defatted with the ethanol on the shaker at the material:liquid ratio of 1:25, at the temperature of approximately 60° C., and for the time period of approximately 3 hours. A single factor test may then be performed at a pH of 13. The protein content in the supernatant may then be measured as 62.30%.

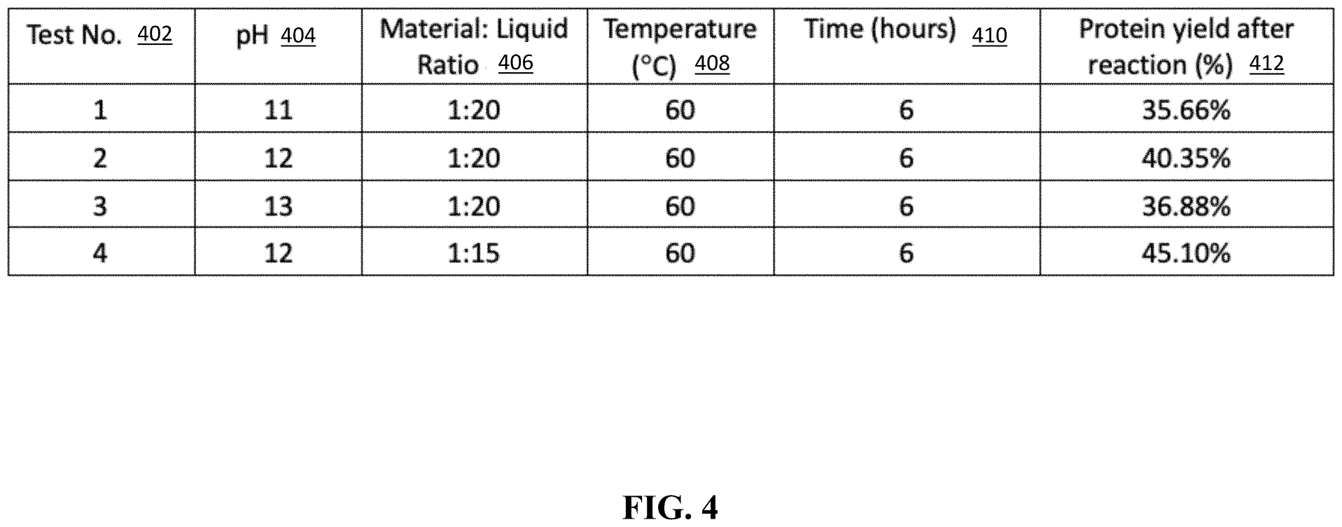

FIG. 4 depicts tabular results of reducing a material:liquid ratio and extending a reaction time from FIG. 3, according to at least some embodiments described herein. The columns of FIG. 4 include: a test number column 402, a pH column 404, a material:liquid ratio column 406, a temperature column 408 (which is measured in ° C.), a reaction time period column 410 (which is measured in hours), and a protein yield column 412 (which is measured as a % after the reaction).

As shown in FIG. 4, the material:liquid ratio is reduced from the range in FIG. 3 of 1:25 to 1:50 to a range of 1:15 to 1:20 in FIG. 4. Moreover, as shown in FIG. 4, the reaction time of reaction time period column 410 is extended from 3 hours (in FIG. 3) to 6 hours. As an illustrative example (described as Test 4), the chlorella may be defatted with the ethanol on the shaker at the material:liquid ratio of 1:15, at the temperature of approximately 60° C., and for the time period of approximately 6 hours. A single factor test may then be performed at a pH of 12. The protein content in the supernatant may then be measured as 45.10%.

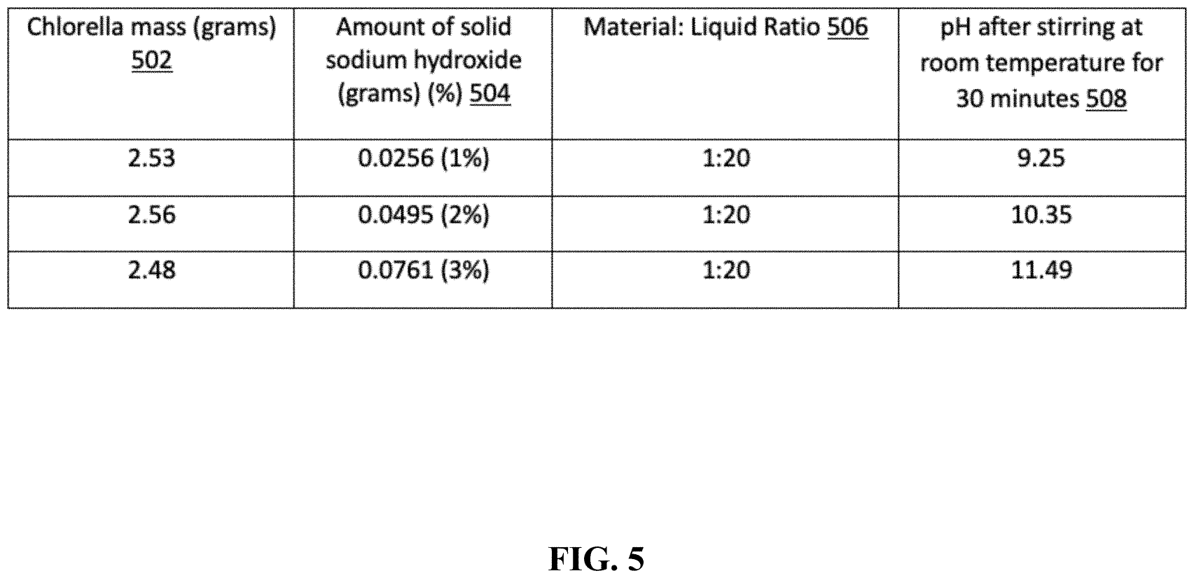

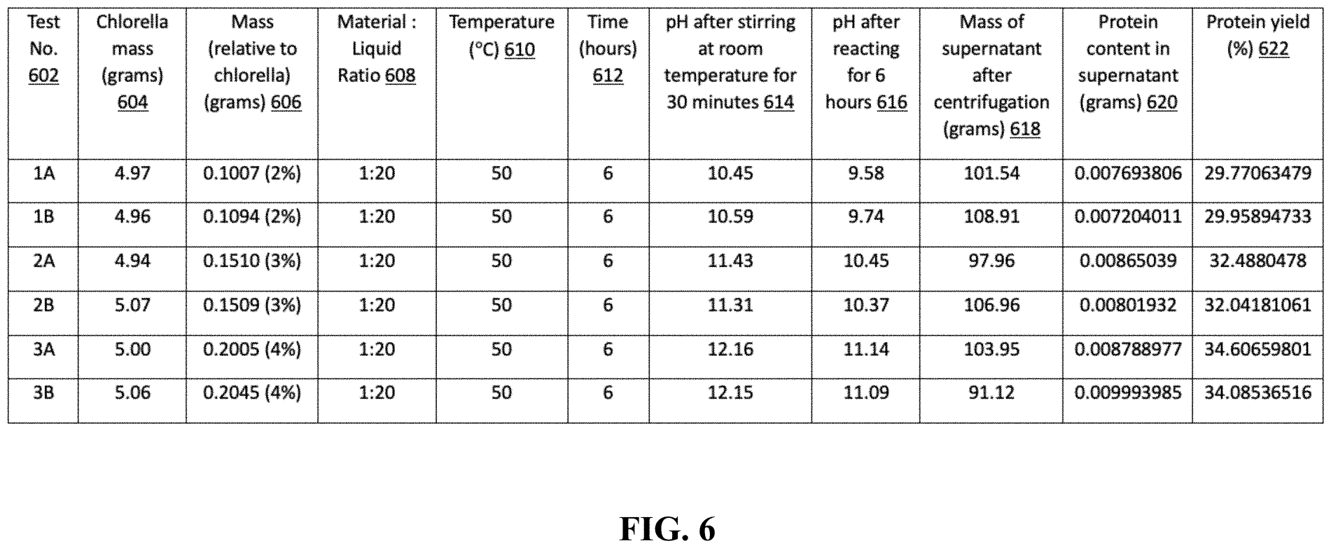

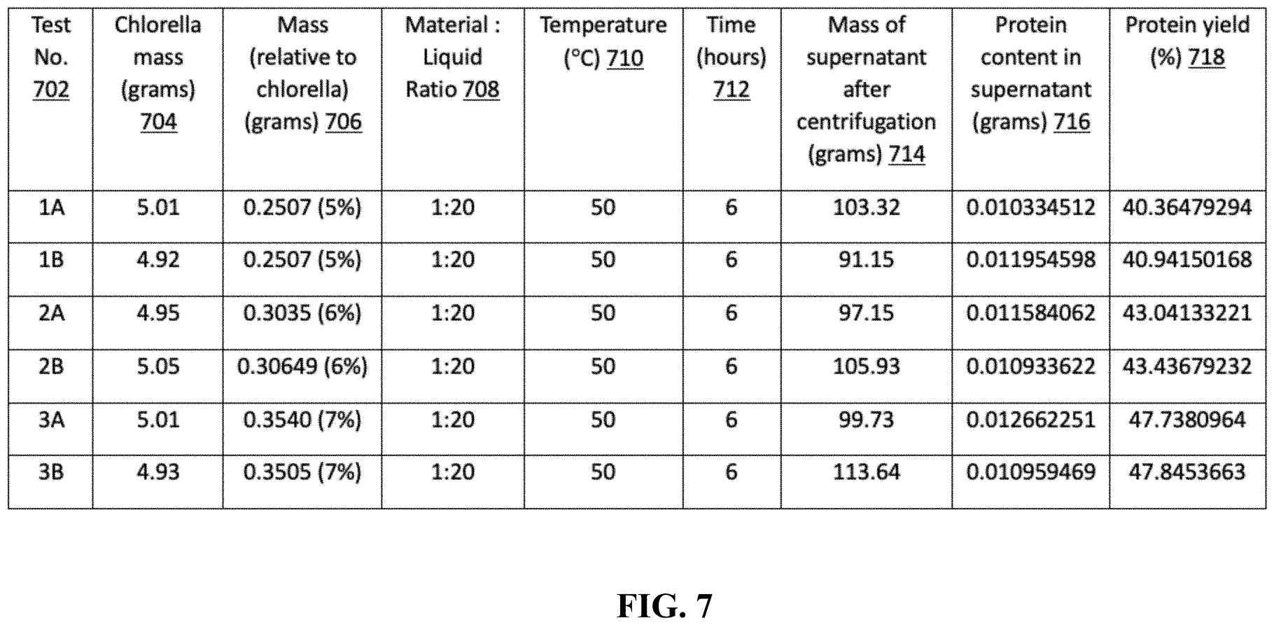

FIG. 5, FIG. 6, and FIG. 7 depict tabular results of obtaining a supernatant following centrifugation, according to at least some embodiments described herein.

The columns of FIG. 5 include: a chlorella mass column 502 (measured in grams), an amount of solid NaOH column 504 (measured in grams, as well as a % of a weight of the chlorella protein), a material:liquid ratio column 506, and a pH column 508 (associated with a pH after stirring at room temperature for a time period of 30 minutes).

The columns of FIG. 6 include: a test number column 602, a chlorella mass column 604 (measured in grams), a mass relative to the chlorella column 606 (which measures the mass of the solid NaOH in grams, as well as the mass of the solid NaOH in comparison to the chlorella, which may be measured in a %), a material:liquid ratio column 608, a temperature column 610 (which is measured in ° C.), a first pH column 614 (which measures the pH after stirring the mixture at room temperature for 30 minutes), a second pH column 616 (which measures the pH after reacting the mixture for 6 hours), a mass of supernatant column 618 (which measures the mass of the supernatant in grams after centrifugation), a protein content column 620 (which measures the protein content in the supernatant in grams), and a protein yield column 622 (which measures the protein yield as a %).

The columns of FIG. 7 include: a test number column 702, a chlorella mass column 704 (measured in grams), a mass relative to the chlorella column 706 (which measures the mass of the solid NaOH in grams, as well as the mass of the solid NaOH in comparison to the chlorella, which may be measured in a %), a material:liquid ratio column 708, a temperature column 710 (which is measured in ° C.), a time period column 712 (measured in hours), a mass of supernatant column 714 (which measures the mass of the supernatant in grams after centrifugation), a protein content column 716 (which measures the protein content in the supernatant in grams), and a protein yield column 718 (which measures the protein yield as a %).

To obtain the supernatant, as shown in FIG. 5, FIG. 6, and FIG. 7, the following process steps occur: chlorella is added with solid NaOH. A mass of the chlorella may be in a range between approximately 2.48 grams to approximately 5.06 grams. The amount of solid NaOH may be in a range between approximately 0.0256 grams to approximately 0.3505 grams (or between 1% to 7% of the weight of the chlorella protein). The material:liquid ratio is approximately 1:20.

After stirring the mixture at room temperature for 30 minutes, the pH is measured, and then the reaction is carried out at 50° C. for 6 hours. The pH may be in a range between a pH of 9.25 and a pH of 11.49. The centrifugation occurs at approximately 800 rpm for approximately 20 minutes to obtain a protein extract solution of the chlorella protein. The supernatant is obtained following the centrifugation. The supernatant may be present in an amount of approximately 0.0072 grams to 0.0127 grams. Then, a protein recovery rate is calculated from the protein extract solution of the chlorella protein via Equation 1. The protein recovery rate may be in a range between approximately 29.77% to approximately 47.84%.

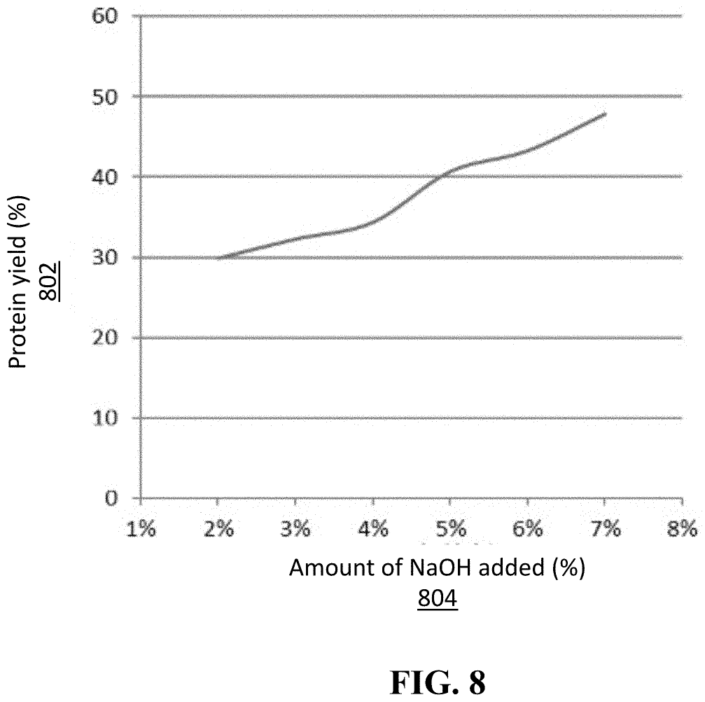

FIG. 8 depicts a graph showing an amount of NaOH added as compared to a protein yield, according to at least some embodiments described herein. A graph of FIG. 8 has an x-axis and a y-axis. The x-axis of the graph showcases an amount of NaOH added as a percentage, in a range from 1% to 8%. The y-axis of the graph showcases a protein yield measured as a percentage, in a range from 0% to 60%. As is shown by the graph, an increase in the NaOH increases the protein yield percentage.

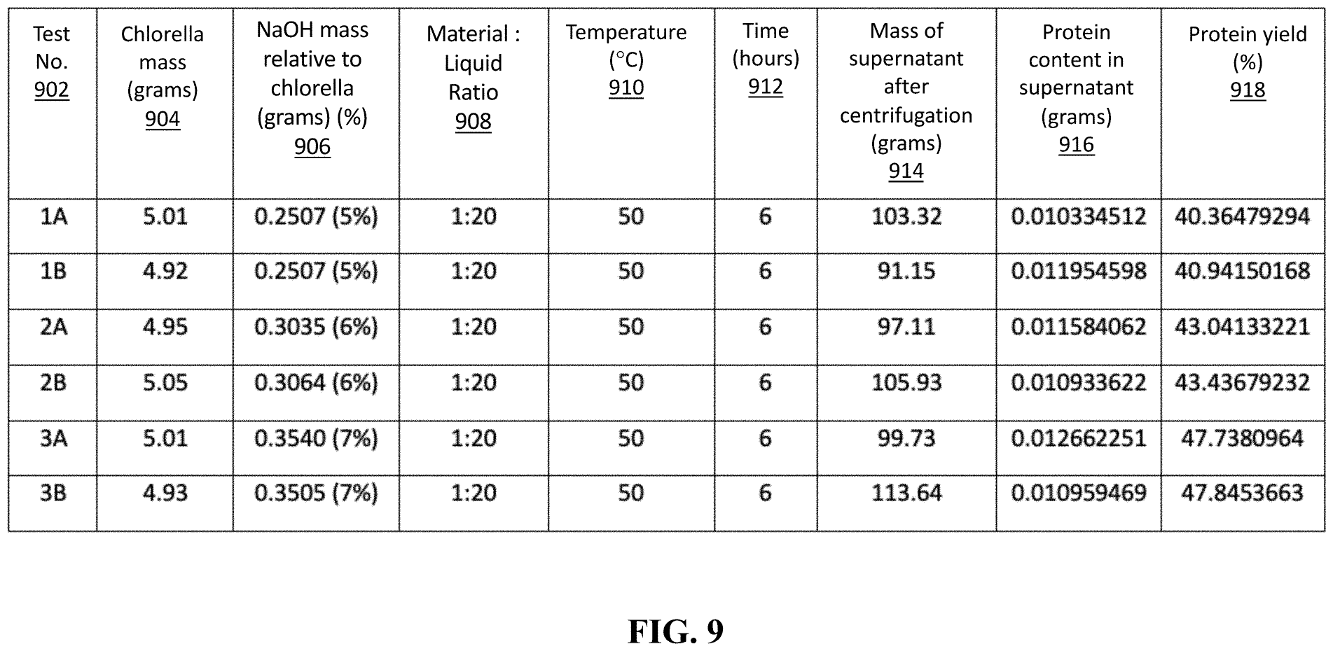

FIG. 9 depicts tabular results of adding an increased amount of solid NaOH to chlorella to increase a protein yield percentage, according to at least some embodiments described herein. The columns of FIG. 9 include, at least, a test number column 902, a chlorella mass column 904 (measured in grams), a mass relative to the chlorella column 906 (which measures the mass of the solid NaOH in grams, as well as the mass of the solid NaOH in comparison to the chlorella, which may be measured in a %), a material:liquid ratio column 908, a temperature column 910 (which is measured in ° C.), a time period column 912 (measured in hours), a mass of supernatant column 914 (which measures the mass of the supernatant in grams after centrifugation), a protein content column 916 (which measures the protein content in the supernatant in grams), and a protein yield column 918 (which measures the protein yield as a %).

The chlorella mass column 904 includes a mass of the chlorella in a range from 4.92 grams to 5.05 grams. The mass relative to the chlorella column 906 includes a range from 0.2507 grams to 0.3505 grams (or 5% to 7%). The material:liquid ratio column 908 includes a 1:20 ratio. The temperature column 910 includes a temperature of approximately 50° C. The time period column 912 is approximately 6 hours. The mass of supernatant column 914 includes a range from 91.15 grams to 105.93 grams. The protein content column 916 includes a range from 0.0103 grams to 0.0127 grams. The protein yield column 918 includes a range from 40.36% to 47.74%. As shown by Test number 3A and Test number 3B, increasing the mass of the NaOH to approximately 0.35 grams or 7% of the weight of the chlorella results in an increase in the protein yield percentage.

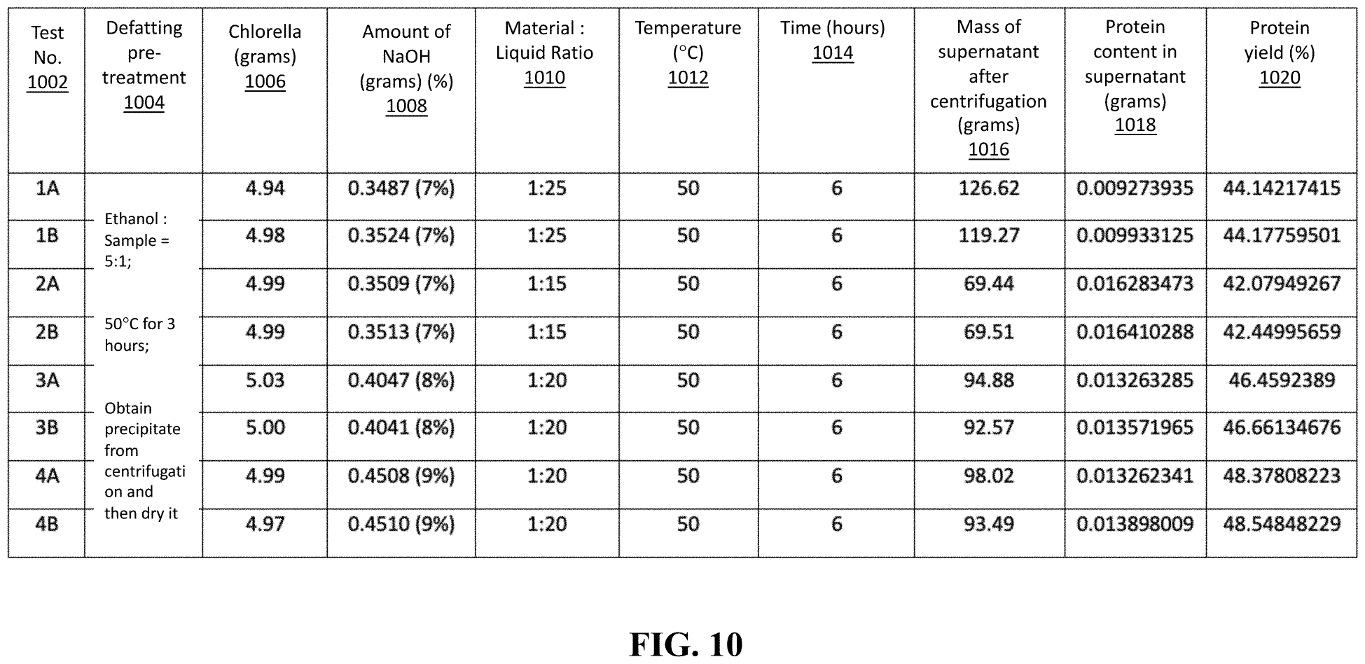

FIG. 10 depicts tabular results of adding an increased amount of solid NaOH to chlorella to increase a protein yield percentage, according to at least some embodiments described herein. The columns of FIG. 10 include, at least, a test number column 1002 and a defatting pre-treatment column 1004. The defatting pre-treatment column 1004 includes numerous process steps that are applied to each test of the test number column 1002. These process steps include: (1) having an ethanol:sample molar ratio of 5:1; (2) reacting the sample at approximately 50° C. for approximately 3 hours; (3) centrifuging the sample; (4) obtaining a precipitate from the centrifugation; and (5) drying the precipitate.

The columns of FIG. 10 may also include: a chlorella mass column 1006 (measured in grams), an amount of NaOH column 1008 (which measures the mass of the solid NaOH in grams, as well as the mass of the solid NaOH in comparison to the chlorella, which may be measured in a %), a material:liquid ratio column 1010, a temperature column 1012 (which is measured in ° C.), a time period column 1014 (measured in hours), a mass of supernatant column 1016 (which measures the mass of the supernatant in grams after centrifugation), a protein content column 1018 (which measures the protein content in the supernatant in grams), and a protein yield column 1020 (which measures the protein yield as a %).

As shown in FIG. 10, the mass of the chlorella may be between approximately 4.94 grams and 5.03 grams and the amount of the NaOH may be between 7% to 9% (as compared to the mass of the chlorella). The mass:liquid ratio is between 1:15 to 1:25. The temperature is approximately 50° C. and the time period is approximately 6 hours. The mass of the supernatant after the centrifugation is between 69.44 grams to 126.62 grams. The protein content in the supernatant is between 0.0093 grams and 0.0139 grams. The protein yield percentage is between 42.08% and 48.55%.

As shown in FIG. 9, the amount of NaOH, as compared to the weight of the chlorella was present in a range between 5% to 7%. FIG. 10 increases the amount of the NaOH, as compared to the weight of the chlorella, to being in a range between 7% to 9%. Specifically, in FIG. 10, Test number 4A and Test number 4B include 9% of NaOH and result in protein yield percentages of at least 48%.

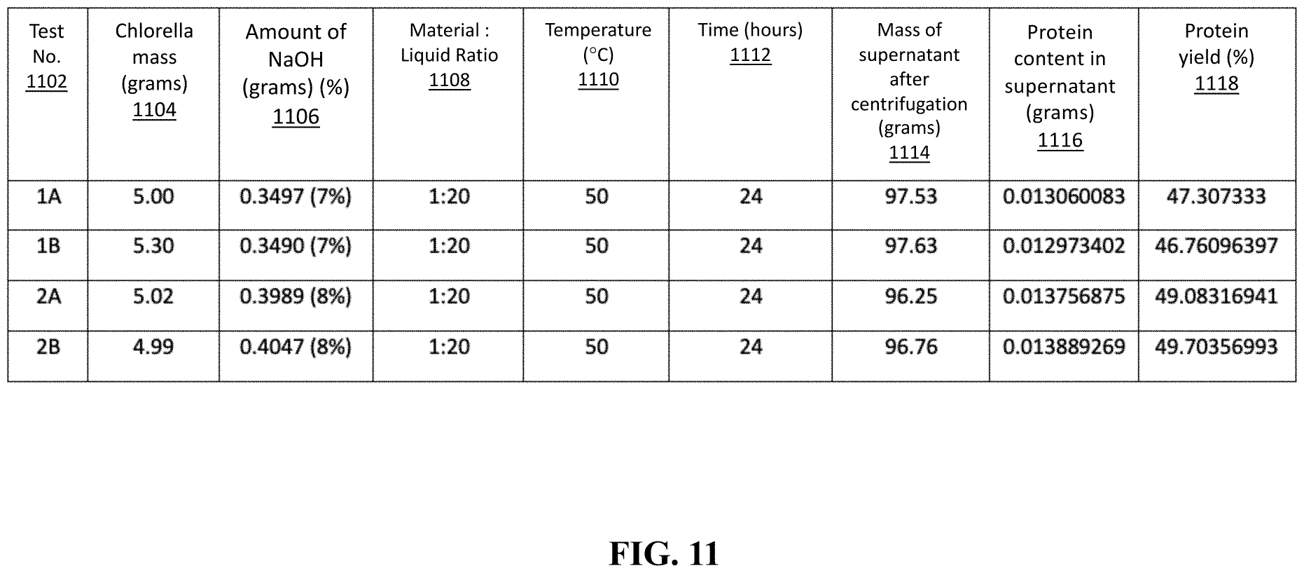

FIG. 11 depicts tabular results of increasing the reaction time period from FIG. 10, according to at least some embodiments described herein. The columns of FIG. 11 include, at least, a test number column 1102, a chlorella mass column 1104 (measured in grams), an amount of NaOH column 1106 (which measures the mass of the solid NaOH in grams, as well as the mass of the solid NaOH in comparison to the chlorella, which may be measured in a %), a material:liquid ratio column 1108, a temperature column 1110 (which is measured in ° C.), a time period column 1112 (measured in hours), a mass of supernatant column 1114 (which measures the mass of the supernatant in grams after centrifugation), a protein content column 1116 (which measures the protein content in the supernatant in grams), and a protein yield column 1118 (which measures the protein yield as a %).

In FIG. 10, the material:liquid ratio is present in a range between 1:15 to 1:25 and the reaction time period was approximately 6 hours. In FIG. 11, the material:liquid ratio is 1:20 and the reaction time period is approximately 24 hours. Test 2B shows that adding 8% NaOH under these conditions yields a protein yield percentage of approximately 49.70%.

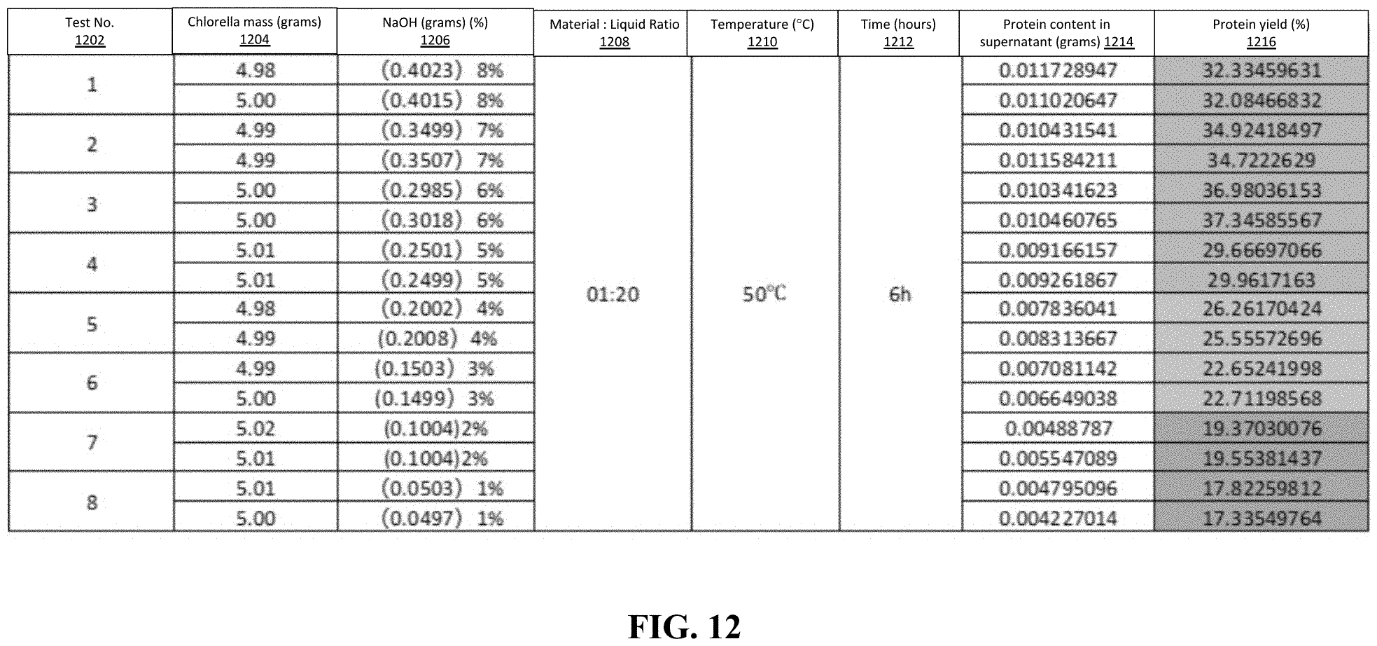

FIG. 12 depicts tabular results of a protein yield percentage from varying an amount of sodium hydroxide in a range between 1% to 8%, according to at least some embodiments described herein. The columns of FIG. 12 include, at least, a test number column 1202, a chlorella mass column 1204 (measured in grams), an amount of NaOH column 1206 (which measures the mass of the solid NaOH in grams, as well as the mass of the solid NaOH in comparison to the chlorella, which may be measured in a %), a material:liquid ratio column 1208, a temperature column 1210 (which is measured in ° C.), a time period column 1212 (measured in hours), a protein content column 1214 (which measures the protein content in the supernatant in grams), and a protein yield column 1216 (which measures the protein yield as a %).

As shown in FIG. 12, the material:liquid ratio is 1:20, the temperature is approximately 50° C., and the reaction time is approximately 6 hours. The amount of NaOH varies in a range between 1% to 8%, as compared to the mass of the chlorella. As can be seen in Test 1, a higher amount of NaOH results in a higher protein yield percentage.

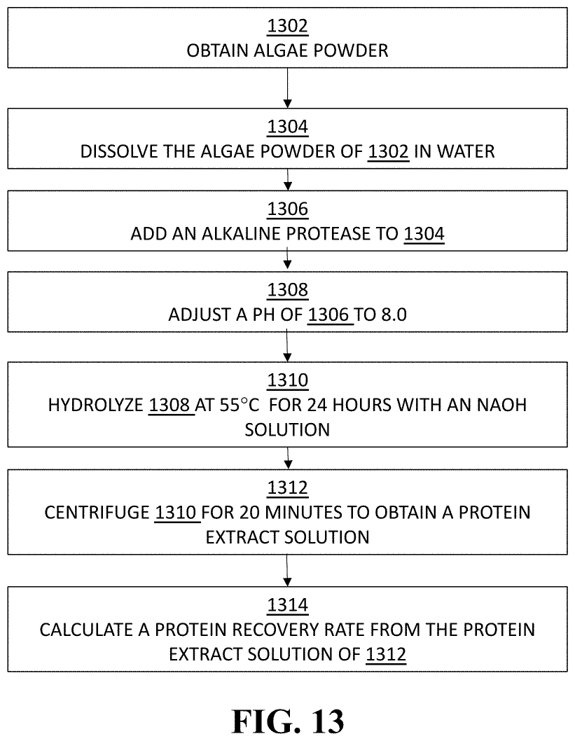

FIG. 13 depicts a schematic block diagram of an enzyme extraction method for chlorella protein, according to at least some embodiments described herein.

The method of FIG. 13 begins at a process step 1302, which includes obtaining algae powder. The process step 1302 is followed by a process step 1304, where approximately 25.0 grams of algae powder is dissolved in approximately 375 mL of water. The process step 1304 is followed by a process step 1306, where an alkaline protease is added to the solution of the process step 1304. The alkaline protease may be added in an amount between 0.01% to 0.2%. In examples, the alkaline protease is cellulase, pectinase, or alkaline protease 37071. However the alkaline protease is not limited to these examples provided herein.

The process step 1306 is followed by a process step 1308, where a pH of the solution in the process step 1306 is adjusted to a pH of 8.0. The process step 1308 is followed by a process step 1310, where the solution of the process step 1308 is hydrolyzed at 55° C. for approximately 24 hours with an NaOH solution. The process step 1310 is followed by a process step 1312, where the solution of the process step 1310 is centrifuged for approximately 20 minutes to obtain a protein extract solution. The process step 1312 is followed by a process step 1314, where a protein recovery rate is calculated from the protein extract solution for the process step 1312. The process step 1314 may be followed by additional process steps including: extracting the chlorella and drying the chlorella.

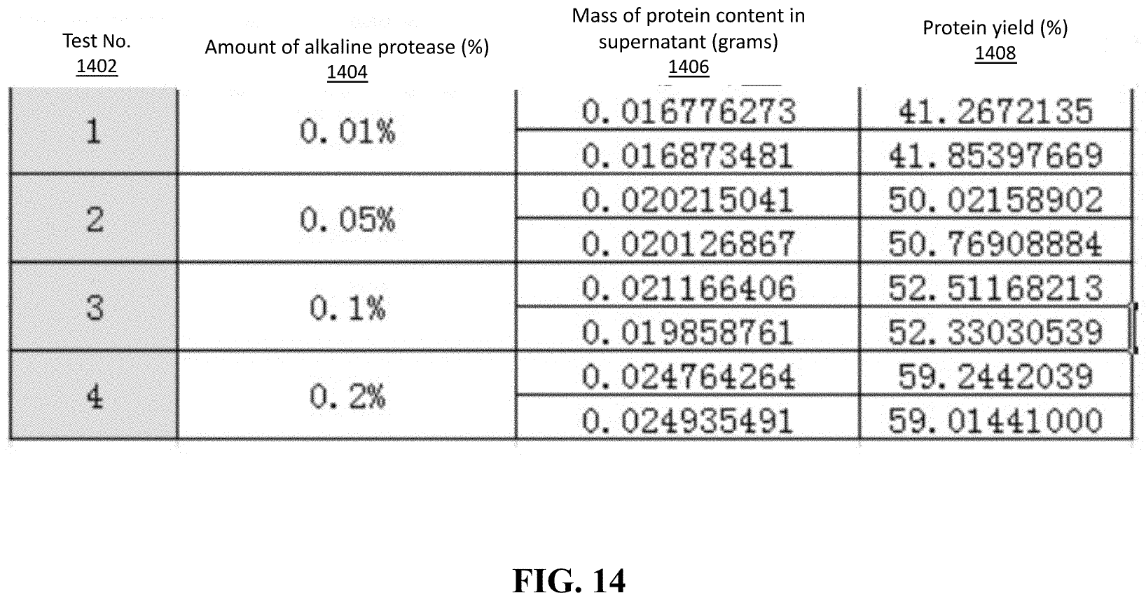

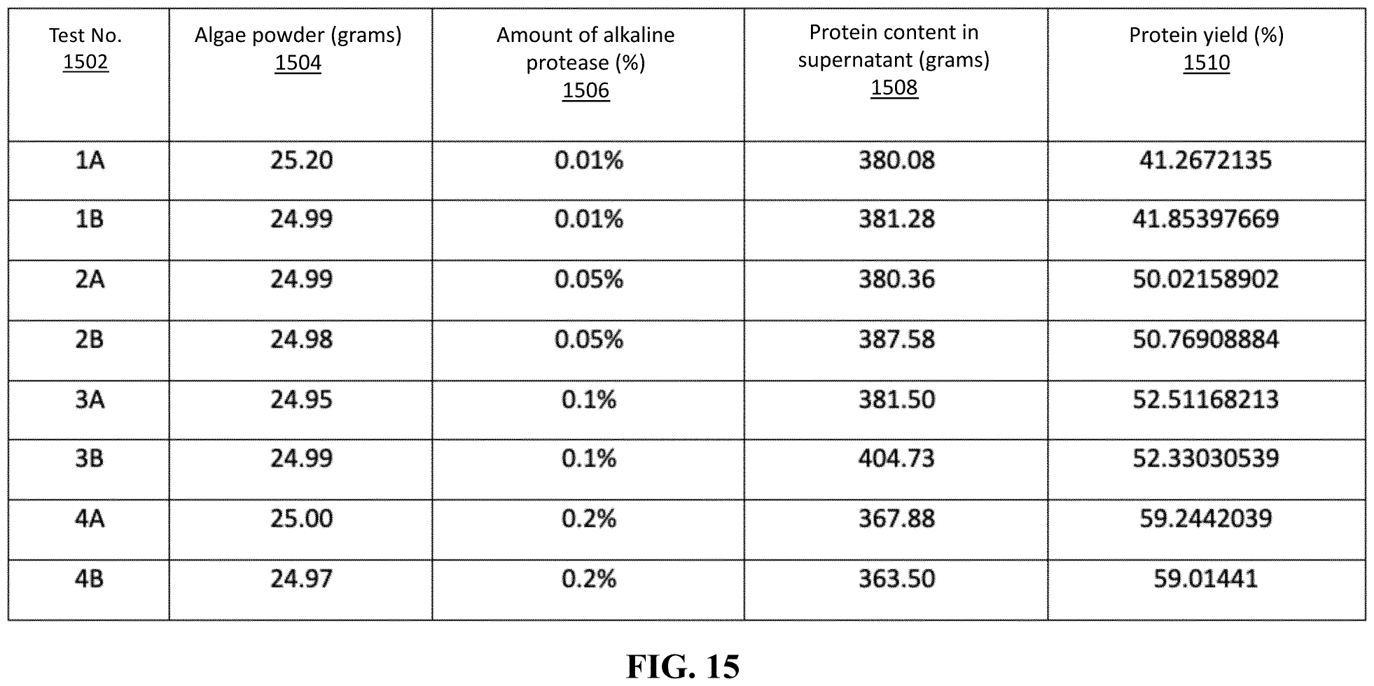

FIG. 14 and FIG. 15 depict tabular results of the enzyme extraction method of FIG. 13, according to at least some embodiments described herein.

FIG. 14 includes, at least, the following columns: a test number column 1402, an amount of an alkaline protease column 1404 (measured as a percentage), a mass of a protein content in a supernatant column 1406 (measured in grams), and a protein yield column 1408 (measured as a percentage). As shown, the amount of the alkaline protease ranges from 0.01% to 0.2%. The mass of the protein content in the supernatant ranges from 0.0168 grams to 0.0249 grams. The protein yield ranges from 41.26% to 59.24%. As shown in Test number 4, the higher the amount of the alkaline protease correlates to a higher protein yield percentage.

FIG. 15 includes similar columns, such as: a test number column 1502, a mass of an algae powder column 1504 (in grams), an amount of an alkaline protease column 1506 (measured as a percentage), a mass of a protein content in a supernatant column 1508 (measured in grams), and a protein yield column 1510 (measured as a percentage). As shown in FIG. 15, the algae powder is present in a range of approximately 24.95 grams to approximately 25.20 grams. The alkaline protease is present in a range of approximately 0.01% to 0.2%. As can be seen in Test number 4A and Test number 4B, the greater the amount of the alkaline protease (e.g., 0.2%), the higher the protein yield percentage (e.g., between 59.01% and 59.24%).

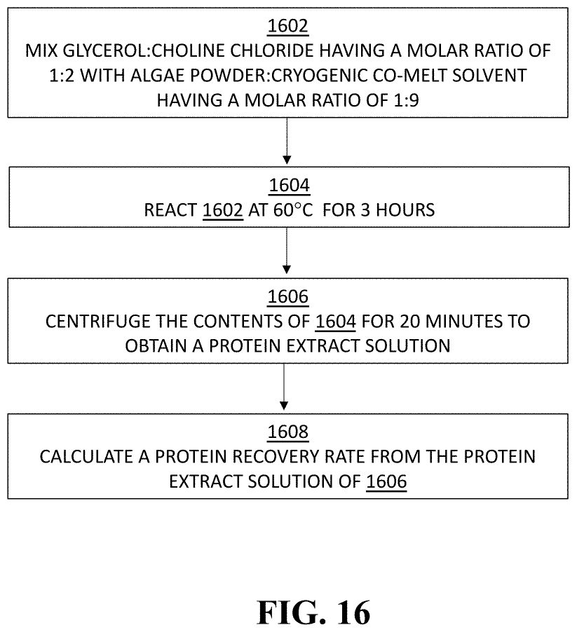

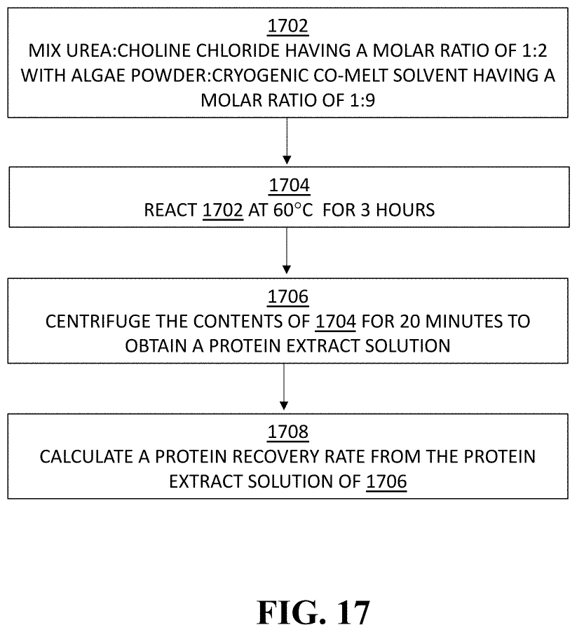

FIG. 16 and FIG. 17 depict schematic block diagrams of a first and a second low-temperature deep eutectic solvent (DES) extraction method for chlorella protein, according to at least some embodiments described herein.

DES is a stable solvent formed by the combination of two or three substances by hydrogen bonds between molecules. The composition of DES interacts with a protein (e.g. hydrogen bonding), extracts the protein from raw material, and then separates the protein by washing or alcohol. DES raw materials have a low cost, are easy to biodegrade, and provide better environmental compatibility.

A first method of FIG. 16 begins with a process step 1602, which includes mixing a eutectic solvent (e.g., glycerol:choline chloride having a molar ratio of 1:2) with algae powder:cryogenic co-melt solvent having a molar ratio of 1:9. It should be appreciated that other eutectic solvents may be used.

The process step 1602 is followed by a process step 1604, where the solution of the process step 1602 is reacted at approximately 60° C. for approximately 3 hours. The process step 1604 is followed by a process step 1606, where the contents of the process step 1604 are centrifuged for approximately 20 minutes to obtain a protein extract solution. The process step 1606 is followed by a process step 1608, where a protein recovery rate is calculated from the protein extract solution of the process step 1606.

A second method of FIG. 17 begins with a process step 1702, which includes mixing a eutectic solvent (e.g., urea:choline chloride having a molar ratio of 1:2) with algae powder:cryogenic co-melt solvent having a molar ratio of 1:9. The process step 1702 is followed by a process step 1704, where the solution of the process step 1702 is reacted at approximately 60° C. for approximately 3 hours. The process step 1704 is followed by a process step 1706, where the contents of the process step 1704 are centrifuged for approximately 20 minutes to obtain a protein extract solution. The process step 1706 is followed by a process step 1708, where a protein recovery rate is calculated from the protein extract solution of the process step 1706.

It should be appreciated that in some examples, prior to the process step 1602 or the process step 1702, the sample is defatted, according to the method described herein.

When introducing elements of the present disclosure or the embodiments thereof, the articles “a,” “an,” and “the” are intended to mean that there are one or more of the elements. Similarly, the adjective “another,” when used to introduce an element, is intended to mean one or more elements. The terms “including” and “having” are intended to be inclusive such that there may be additional elements other than the listed elements.

Although this invention has been described with a certain degree of particularity, it is to be understood that the present disclosure has been made only by way of illustration and that numerous changes in the details of construction and arrangement of parts may be resorted to without departing from the spirit and the scope of the invention.

What is claimed is:

1. An extraction method for chlorella protein, the method comprising:
adding an alkaline solution to algae powder to form a mixture;
extracting the chlorella protein from the mixture at a temperature for a first time period;
centrifuging the mixture for a second time period to obtain a protein extract solution of the chlorella protein; and
calculating a protein recovery rate from the protein extract solution of the chlorella protein.

2. The extraction method of claim 1, wherein the alkaline solution is added in a range between approximately 1% to approximately 8% of a weight of the chlorella protein.

3. The extraction method of claim 1, wherein the algae powder comprises a protein content in a range of approximately 60% to approximately 65%.

4. The extraction method of claim 3, wherein the algae powder comprises a protein content of approximately 61.51%.

5. The extraction method of claim 1, wherein the temperature is approximately 50° C.

6. The extraction method of claim 1,
wherein the first time period is approximately 6 hours, and
wherein the second time period is approximately 20 minutes.

7. The extraction method of claim 1, wherein the centrifuging the mixture for the second time period to obtain the protein extract solution of the chlorella protein occurs at approximately 800 rpm.

8. The extraction method of claim 1, wherein the protein recovery rate of the protein extract solution is calculated by:
$$Protein recovery rate / \% \frac{Supernatant protein content \times supernatant mass}{Chlorella mass \times algal powder protein content} \times 100$$

9. The extraction method of claim 1, wherein the alkaline solution is a sodium hydroxide (NaOH) solution.

10. The extraction method of claim 1, wherein the algae powder is present in a range of approximately 4.98 grams to approximately 5.02 grams.

11. An enzyme extraction method for chlorella protein, the method comprising:
dissolving an algae powder in water to form a solution;
adding an alkaline protease to the solution;
adjusting a pH of the solution to the pH of 8.0;
hydrolyzing the solution at a temperature with an alkaline solution for a first time period;
centrifuging the mixture for a second time period to obtain a protein extract solution of the chlorella protein; and
calculating a protein recovery rate from the protein extract solution of the chlorella protein.

12. The enzyme extraction method of claim 11, wherein the algae powder is present in approximately 25.0 grams.

13. The enzyme extraction method of claim 11, wherein the water is present in approximately 375 mL.

14. The enzyme extraction method of claim 11, wherein the alkaline protease is present in a range of approximately 0.01% to approximately 0.2%.

15. The enzyme extraction method of claim 11, wherein the temperature is approximately 55° C.

16. The enzyme extraction method of claim 12, wherein the first time period is 24 hours, and wherein the second time period is 20 minutes.

17. A low-temperature deep eutectic solvent (DES) extraction method of chlorella protein, the method comprising:
adding a first material:a second material having molar ratios of 1:2 to an algae powder:cryogenic co-melt solvent having molar ratios of 1:9 to form a mixture;
reacting the mixture at a temperature for a first time period;
centrifuging the mixture for a second time period to obtain a protein extract solution of the chlorella protein; and
calculating a protein recovery rate from the protein extract solution of the chlorella protein.

18. The low-temperature DES extraction method of claim 17, wherein the first material:second material is glycerol:choline chloride.

19. The low-temperature DES extraction method of claim 17, wherein the first material:second material is urea:choline chloride.

20. The low-temperature DES extraction method of claim 17,
wherein the temperature is approximately 60° C.,
wherein the first time period is approximately 3 hours, and
wherein the second time period is approximately 20 minutes.
